(19) United States
(12) Patent Application Publication
(10) Pub. No.: US 2024/0197953 A1
(43) Pub. Date: Jun. 20, 2024

(54) DEVICES AND METHODS FOR ENDOSCOPIC PLURALITY STRIP DELIVERY

(71) Applicant: Boston Scientific Scimed, Inc., Maple Grove, MN (US)

(72) Inventors: Lauren S. LYDECKER, Millbury, MA (US); Megan CHROBAK, Westford, MA (US); Eric GREENE, Wayland, MA (US); Paul SMITH, Smithfield, RI (US)

(73) Assignee: Boston Scientific Scimed, Inc., Maple Grove, MN (US)

(21) Appl. No.: 18/530,530

(22) Filed: Dec. 6, 2023

Related U.S. Application Data
(60) Provisional application No. 63/431,075, filed on Dec. 8, 2022.

(51) Int. Cl. A61L 24/08; A61B 17/08

(52) U.S. Cl. CPC A61L 24/08; A61B 17/085; A61B 2017/00818; A61L 2400/04

(57) ABSTRACT

Devices, kits, and methods of delivering a plurality of strips to a tissue defect site to promote covering and/or closure of the tissue defect are described. The kit may include a first medical device configured to push the plurality of strips housed therein through a distal opening and a second medical device defining a working channel for insertion of the first medical device. Methods may include introducing a distal portion of a medical device housing a plurality of strips that include a biocompatible material into a gastrointestinal tract, positioning the distal portion of the device proximate a target site of a tissue wall of the gastrointestinal tract that includes a defect, and applying the plurality of strips to the target site by pushing the plurality of strips out of a distal opening of the device.

### CROSS-REFERENCE TO RELATED APPLICATIONS

This application claims the benefit of priority to U.S. Provisional Application No. 63/431,075, filed on Dec. 8, 2022, which is incorporated herein by reference in its entirety.

### TECHNICAL FIELD

The present disclosure relates generally to medical devices, kits, and related methods of use thereof. More particularly, the present disclosure includes devices, kits, and methods useful in endoscopic medical procedures, such as applying a plurality of strips to a tissue defect site to promote covering and/or closure of the tissue defect.

### BACKGROUND

Various medical procedures are used for treatment of tissue. For example, an endoscopic procedure of the gastrointestinal (GI) tract or other organ systems may be performed for interventional and/or therapeutic purposes, such as treating issues below the mucosa, removing pre-cancerous mucosal tissue or tumors, and removing lesions. Such procedures may result in perforation, post-surgical leaks, or other wounds in need of repair. Internal wounds or tissue defects such as, e.g., inflammation, ulcerations, and the like, can result naturally and likewise benefit from therapeutic treatment. To treat internal wounds, agents, such as hemostatic agents, may be applied via medical devices to a target site. For example, hemostatic agent particles may be sprayed onto a wound or tissue defect. However, applying hemostatic agents, e.g., via spraying, to an internal tissue defect may present particular challenges, including potential distribution through the defect and into an unwanted part of the body.

### SUMMARY

The present disclosure includes medical devices, kits, and methods of use thereof, e.g., methods of applying a plurality of strips to a tissue defect site to promote covering and/or closure of the tissue defect. For example, the present disclosure includes a kit that includes a first medical device defining a lumen that houses a shaft and a chamber proximate a distal end of the first device, wherein the chamber houses a plurality of strips comprising a biocompatible material, and the shaft is moveable along the lumen to push the plurality of strips through a distal opening of the first device. The kit also includes a second medical device defining a working channel, the first device being insertable into the working channel. In some examples herein, the biocompatible material comprises chitosan, cellulose, poly(2-hydroxyethyl methacrylate), polystyrene, collagen, gelatin, fibrin, polyethylene glycol (PEG), hyaluronic acid, block copolymers, or a combination thereof. In some examples, the plurality of strips comprise chitosan or derivative thereof, such as thiolated chitosan, chitosan crosslinked with tripolyphosphate, and/or carboxymethylcellulose chitosan crosslinked with PEGamine. The plurality of strips may be bioresorbable. According to some aspects of the present disclosure, the second device may be an endoscope.

Additionally or alternatively, each strip of the plurality of strips may have a length ranging from about 5 mm to about 40 mm, a width ranging from about 1.5 mm to about 5 mm, and/or a thickness ranging from about 1 μm to about 100 μm. In some examples, the plurality of strips may include strips of different lengths and/or widths. According to some aspects of the present disclosure, the strips of the plurality strips may be planar. In at least one example, the plurality of strips includes at least one strip having a variable thickness along the length of the at least one strip. With respect to the first device, for example, the shaft may include a distal end having cross-sectional dimension larger than a cross-sectional dimension of the shaft, such that the distal end of the shaft contacts and slides along walls of the lumen as the shaft moves along the lumen. The kit may further comprise a balloon device.

The present disclosure also includes use of the kit to treat a target site of tissue. For example, the second device may be an endoscope and the tissue may be tissue of the gastrointestinal tract. In some examples, the target site may include a tissue defect, and the plurality of strips may fill, cover, and/or patch the tissue defect. In at least one example, with respect to use of the kit, the plurality of strips may change from a planar configuration when housed within the first device to a curled configuration when applied to the target site.

The present disclosure also includes a method of treating a subject, the method comprising: introducing a distal portion of a medical device into a gastrointestinal tract of the subject, wherein the distal portion houses a plurality of strips comprising a biocompatible material; positioning the distal portion of the device proximate a target site of a tissue wall of the gastrointestinal tract that includes a defect; and applying the plurality of strips to the target site by pushing the plurality of strips out of a distal opening of the device, wherein the plurality of strips at least partially fill the defect or form a layer over the defect. The biocompatible material may comprise chitosan, cellulose, poly(2-hydroxyethyl methacrylate), polystyrene, collagen, gelatin, fibrin, polyethylene glycol (PEG), hyaluronic acid, a block copolymer, or a combination thereof. For example, the biocompatible material may comprise thiolated chitosan, chitosan crosslinked with tripolyphosphate, or carboxymethylcellulose chitosan crosslinked with PEGamine. In some examples, the biocompatible material may be bioresorbable.

The method may further comprise applying a therapeutic agent to the target site after applying the plurality of strips to the target site. According to some aspects of the present disclosure, the therapeutic agent may be a hemostatic agent. In some examples herein, applying the therapeutic agent may comprise spraying the therapeutic agent onto the target site. In some examples, pushing the plurality of strips out of the distal opening of the device may include advancing a shaft of the device distally to push the plurality of strips with a distal end of the shaft. In such examples, the distal end of the shaft may have a cross-sectional dimension larger than a cross-sectional dimension of the shaft, such that the distal end of the shaft contacts and slides along walls of a lumen as the shaft moves along the lumen. According to some aspects of the present disclosure, each strip of the plurality of strips may have a length ranging from about 5 mm to about 40 mm, a width ranging from about 1.5 mm to about 5 mm, or a thickness ranging from about 1 μm to about 100 μm.

The method may further include applying a biocompatible liquid or a balloon to the plurality of strips after applying the plurality of strips to the target site. In some examples, the plurality of strips may change from a planar configuration when housed within the device to a curled configuration when applied to the target site. In at least one example, the plurality of strips may include at least one strip having a variable thickness along a length of the at least one strip.

Also disclosed herein is a method of treating a subject, the method comprising: introducing a distal portion of a medical device into a gastrointestinal tract of the subject, wherein the distal portion of the device defines a lumen that houses a shaft and a chamber proximate a distal end of the device, wherein the chamber houses a plurality of strips comprising a biocompatible material; positioning the distal portion of the device proximate a target site of a tissue wall that includes a defect; advancing the shaft distally to push the plurality of strips out of a distal opening of the device and onto the target site; and applying a biocompatible liquid, a therapeutic agent, or a balloon to the target site after applying the plurality of strips. The biocompatible material may comprise chitosan, cellulose, poly(2-hydroxyethyl methacrylate), polystyrene, collagen, gelatin, fibrin, polyethylene glycol (PEG), hyaluronic acid, a block copolymer, or a combination thereof. In some examples, the biocompatible material may be bioresorbable, a hemostatic material, or both. For example, the method may include applying a therapeutic agent to the target site after applying the plurality of strips, the therapeutic agent being a hemostatic agent.

The present disclosure also includes a method of treating a subject, the method comprising: introducing a distal portion of a medical device into a gastrointestinal tract of the subject, wherein the distal portion of the device includes a chamber that houses a plurality of strips comprising chitosan or derivative thereof; positioning the distal portion of the device proximate a target site of a tissue wall of the gastrointestinal tract that includes defect; and applying the plurality of strips to the target site by pushing the plurality of strips out of a distal opening of the device; wherein each strip of the plurality of strips has a variable thickness along a length of the strip, and the plurality of strips change from a planar configuration when housed within the device to a curled configuration when applied to the target site. The method may further include applying a biocompatible liquid, a therapeutic agent, or a balloon to the target site after applying the plurality of strips to the target site. For example, the method may include spraying water or an aqueous solution having an acidic pH onto the target site after applying the plurality of strips to the target site.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1A shows an exemplary device for delivering a plurality of strips to a target site, in accordance with some aspects of the present disclosure.

FIGS. 1B-1C show components of the device of FIG. 1A.

FIG. 2 shows exemplary strip dimensions, in accordance with some aspects of the present disclosure.

FIG. 3 illustrates delivery of a plurality of strips to a target site, in accordance with some aspects of the present disclosure.

FIG. 4 illustrates delivery of a plurality of strips to another target site, in accordance with some aspects of the present disclosure.

FIG. 5 illustrates an exemplary system for adhesion of a plurality of strips to tissue, in accordance with some aspects of the present disclosure.

FIG. 6 illustrates another exemplary system for adhesion of a plurality of strips tissue, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass±5% of a specified amount or value. All ranges are understood to include endpoints, e.g., a diameter ranging from 10 mm to 40 mm, includes 10 mm, 40 mm, and all values between.

Embodiments of the present disclosure include medical devices, kits, and methods for treating a target site, e.g., endoscopic delivery of a plurality of strips to a target tissue site. The methods herein include applying a plurality of strips to a target tissue site, such as a tissue defect, for example, to fill and/or patch the target tissue site. The plurality of strips may be applied to a target tissue site prior to application of an agent, e.g., a hemostatic agent, at the target tissue site. The kits and devices thereof herein may be preloaded with a plurality of strips proximate the distal end for delivery of the plurality of strips to a target tissue site. Upon application to a tissue site, such as a tissue defect, the plurality of strips may cover and/or fill the tissue defect.

The plurality of strips herein may serve as a defect closure and/or a barrier against the distribution of certain agents through a defect and into other parts of the body outside of or beyond the target area. For example, the plurality of strips herein may be used to patch (e.g., close) an opening at the site of a tissue defect or to form a layer over a tissue defect. The layer formed by the plurality of strips may serve as a protective layer, barrier, or covering. The plurality of strips herein may also be used to fill certain defects. In some examples, the plurality of strips may be used to seal perforations. Once applied to a defect, the plurality of strips of the present disclosure may provide a surface or structure for an agent, such as a hemostatic agent, to be applied on. In some examples, the plurality of strips herein may be applied to a junction between suture lines (e.g., as part of an anastomotic resection) to serve as a barrier against leakage by reinforcing and/or sealing of the sutures. Additionally, or alternatively, the application of a plurality of strips onto a defect may inhibit or prevent hemostatic agents and/or other agents that are applied, e.g., via spraying, onto the target site (e.g., tissue defect area) from passing through the defect and onto an unwanted part of the body.

In some examples, the plurality of strips may comprise the same or different material as an agent, e.g., hemostatic agent, subsequently applied to the target tissue site. For example, the plurality of strips may comprise a hemostatic material. In addition to being used to fill and/or patch a defect, a plurality of strips comprising hemostatic material may aid in hemostasis when applied to the target tissue site. The plurality of strips may be used in methods for treating various types of tissues, including wounded or diseased tissue, such as lesions, ulceration, perforations, microperforations, and other sites in need of treatment or repair. In some examples, a plurality of strips as disclosed herein may be used in a multiple step treatment process. For example, in an initial step or set of steps, a plurality of strips may be applied to a tissue defect. Next, a hemostatic agent may be applied at the same site, e.g., over the plurality of strips, to prevent or inhibit bleeding of the tissue. The plurality of strips may be made from the same material as the hemostatic agent subsequently applied or from a different material than the hemostatic agent.

Exemplary sites to which a plurality of strips herein may be applied include, but are not limited to, tissues of the gastrointestinal system such as, e.g., the esophagus, the stomach, the small intestine (e.g., duodenum, jejunum, or ileum), and/or the large intestine (e.g., cecum, colon, rectum, or anal canal). In some examples, a plurality of strips may be applied to a defect in tissues of the gastrointestinal system. The plurality of strips may be applied to a variety of tissue defects, including perforations, microperforations, leaks, and fistulae. A defect may be classified as a partial thickness defect or a full thickness defect based on the depth or thickness of the defect. An exemplary thickness of a defect may range from about 3 mm to about 50 mm. For example, a partial thickness tissue defect may be non-transmural and a full thickness tissue defect may be transmural. A full thickness tissue defect may include a perforation, leak, or fistula. In some examples, an anastomotic resection of the small or large intestine may lead to a risk of leaks between suture lines. The plurality of strips herein may be applied to such junctions to serve as a barrier to areas outside of the target area, for example, prior to the application of an agent. In other examples, the plurality of strips herein may be applied to a fistula, e.g., an intestinal fistula, to serve as a barrier to areas outside of the target area, for example, prior to the application of an agent.

According to aspects of the present disclosure, a plurality of strips may be delivered endoscopically, optionally following, or in conjunction with, a medical procedure such as endoscopic mucosal resection (EMR), endoscopic submucosal dissection (ESD), or tissue biopsy. For example, the plurality of strips may be delivered endoscopically to a target site to fill and/or patch a tissue defect resulting from layers removed via ESD or due to ulceration of the mucosal layer. The plurality of strips may comprise one or more biocompatible materials (including e.g., one or more materials derived from biological materials), which may be at least partially or completely bioresorable. For example, upon application to tissue, at least a portion of each of the plurality of strips may dissolve or degrade over time. Exemplary biocompatible materials suitable for the plurality of strips herein include, but are not limited to, polysaccharides such as chitosan (including thiolated chitosan and other functionalized chitosan or chitosan derivatives), cellulose, poly(2-hydroxyethyl methacrylate) (polyHEMA), polystyrene, collagen, gelatin, thrombin, (including, e.g., thrombin/gelatin), fibrin, polyethylene glycol (PEG), hyaluronic acid, block copolymers, and combinations thereof. Without being bound by theory, it is believed that the use of block copolymers or combinations of these materials may increase the persistence of the overall matrix of the strips and may increase the degradative nature as well as the cohesive and/or adhesive nature of the strips. For example, the type of monomer and copolymer units and arrangement of those units may be selected to provide for desired strength and degradation properties for the strips, depending on the type of target site to be treated. The plurality of strips herein may also be cross-linked, such as via ionic crosslinking or covalent crosslinking, to strengthen the matrix provided by the strips. In some examples, the strips may be prepared from a film or sheet comprising the biocompatible material(s). Each strip may be cut to a desired size. In other examples, strips may be prepared by extruding polymers.

In some examples herein, the plurality of strips comprise chitosan and/or derivatives thereof, including but not limited to thiolated chitosan, PEGylated chitosan, catechol-modified chitosan, quaternary ammonium chitosan, and/or carboxymethylchitosan. Chitosan is a linear polysaccharide formed of glucosamine units derived from chitin, the structural component of crustacean exoskeletons.

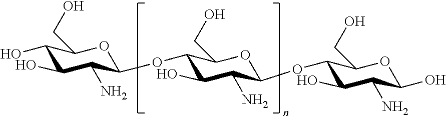

Chitosan is typically prepared by deacetylation of chitin with an alkaline reagent such as sodium hydroxide, yielding a water-soluble material. Chitosan is antimicrobial and has natural bioadhesive and hemostatic properties that allows it to bind to negatively charged surfaces such as mucosal membranes. As discussed further below, in cases in which an agent such as a hemostatic agent is applied to the plurality of strips, the agent may comprise chitosan or derivative thereof.

The plurality of strips herein may comprise chitosan in the form of a salt. For example, salts may be prepared by combining chitosan with a suitable conjugate acid such as acetic acid (forming chitosan acetate) or lactic acid (forming chitosan lactate). Other possible organic acids include, but are not limited to, succinic acid (chitosan succinate), glutamic acid (chitosan glutamate), glycolic acid (chitosan glycolate), and citric acid (chitosan citrate). In an exemplary procedure to prepare a chitosan salt, chitosan is suspended in water at room temperature, followed by the addition of an organic acid (e.g., acetic acid and/or citric acid) to form a gel. The gel is then dried into a film or sheet of desired thickness and dimensions. Without intending to be bound by theory, it is believed that the acid provides for a cross-linked structure. In some examples, the plurality of strips may comprise chitosan crosslinked with tripolyphosphate, e.g., sodium tripolyphosphate, or carboxymethylchitosan crosslinked with PEGamine.

The plurality of strips herein may be formed to have any suitable dimensions, e.g., based on the type of target site, e.g., the size and shape of a tissue defect. In some examples, the length of each individual strip may be on the order of millimeters, e.g., ranging from about 5 mm to about 40 mm, from about 10 mm to about 35 mm, from about 15 mm to about 30 mm, or from about 20 mm to about 25 mm. In some examples, the width of each individual strip may be on the order of millimeters, e.g., ranging from about 1.5 mm to about 5 mm, or from about 2.0 mm to about 4 mm, e.g., a width of about 1.5 mm, about 2.0 mm, about 2.5 mm, about 3 mm, or about 3.5 mm. In some examples, the thickness of each individual strip may be on the order of micrometers, e.g., ranging from about 1 μm to about 300 μm, such as from about 1 μm to about 50 μm, from about 10 μm to about 100 μm, from about 50 μm to about 150 μm, from about 75 μm to about 250 μm, from about 100 μm to about 300 μm, or from about 150 μm to about 275 μm. The thickness of a strip may be uniform or may be variable. For example, a variable thickness may promote curling of the strips upon application to the target site. The strips herein may have a regular geometric shape, such as rectangular or cylindrical, or an irregular shape. In some examples, each individual strip may be capable of changing configuration to fill a tissue defect. According to some aspects, each strip of the plurality of strips may have substantially the same size and/or shape (e.g., the same length, width, and thickness). According to some aspects, the plurality of strips may comprise strips of different sizes and/or shapes from each other (e.g., different lengths, widths, and/or thicknesses).

The plurality of strips may be substantially dry prior to use, e.g., when housed in a delivery device or kit before application to a target site. In some examples herein, the plurality of strips may be moistened after being dispensed from the delivery device or kit. For example, the water or an aqueous solution may be applied to the strips via a fluid channel of an endoscope after the strips are applied to a target site. In some examples, when exposed to moisture (moisture present at the target site and/or applied via an endoscope or other device), each strip may absorb the fluid, causing the strip to swell. According to some aspects of the present disclosure, moistening the plurality of strips in addition to moisture present at the target site may assist in their adhesion to the tissue. In some cases, the plurality of strips may be substantially planar when housed within the device, and may adopt a curled configuration when applied to the target site, optionally once the strips become moistened by liquid present at the target site and/or by a biocompatible liquid such as water, saline solution, or other aqueous solution applied to the target site after the plurality of strips are applied.

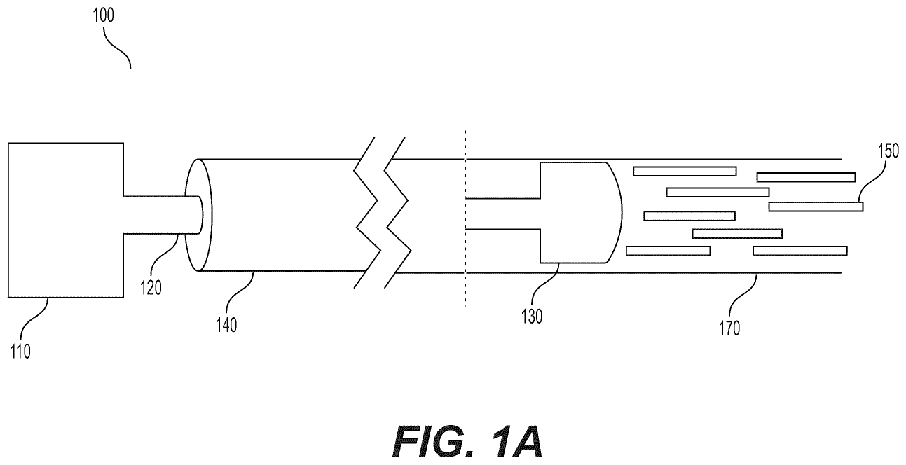

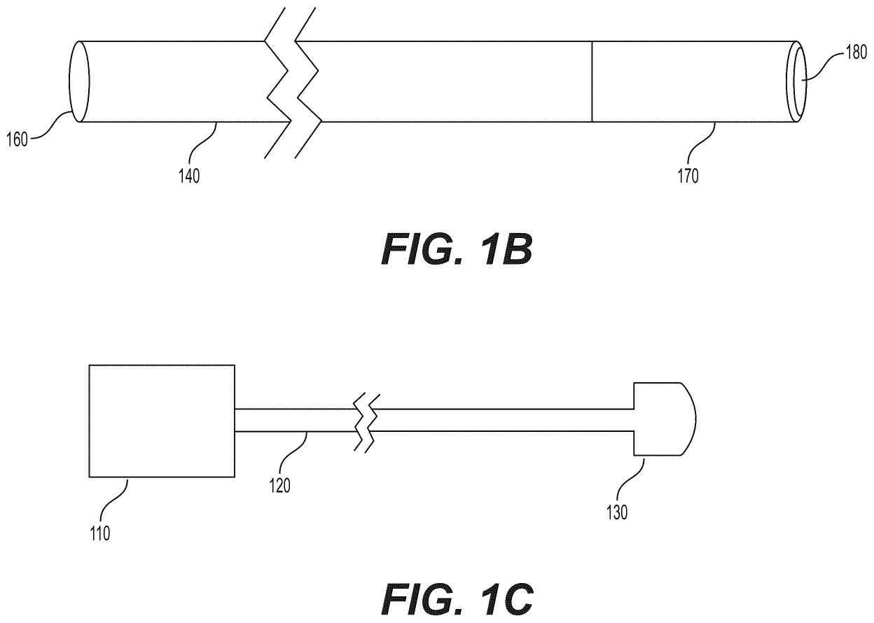

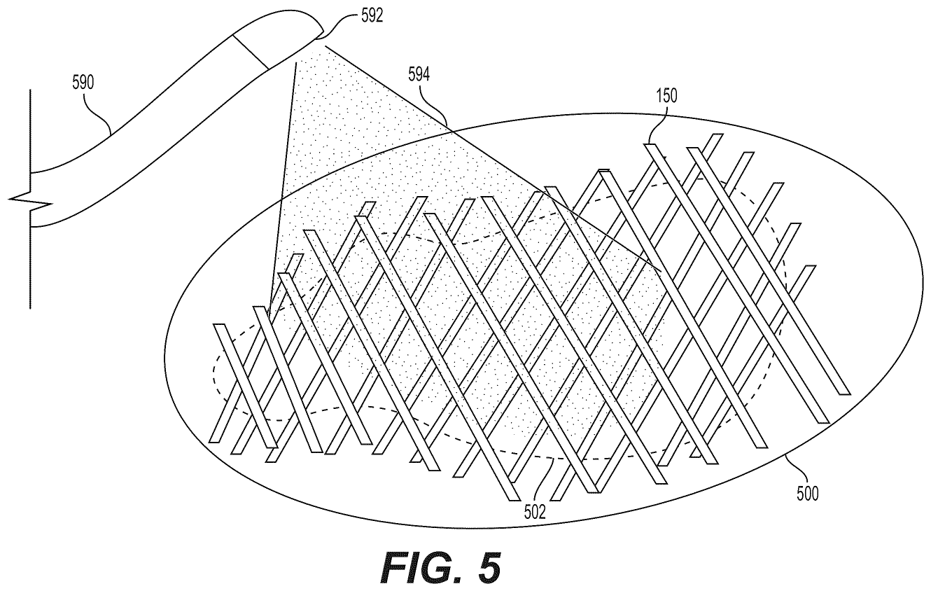

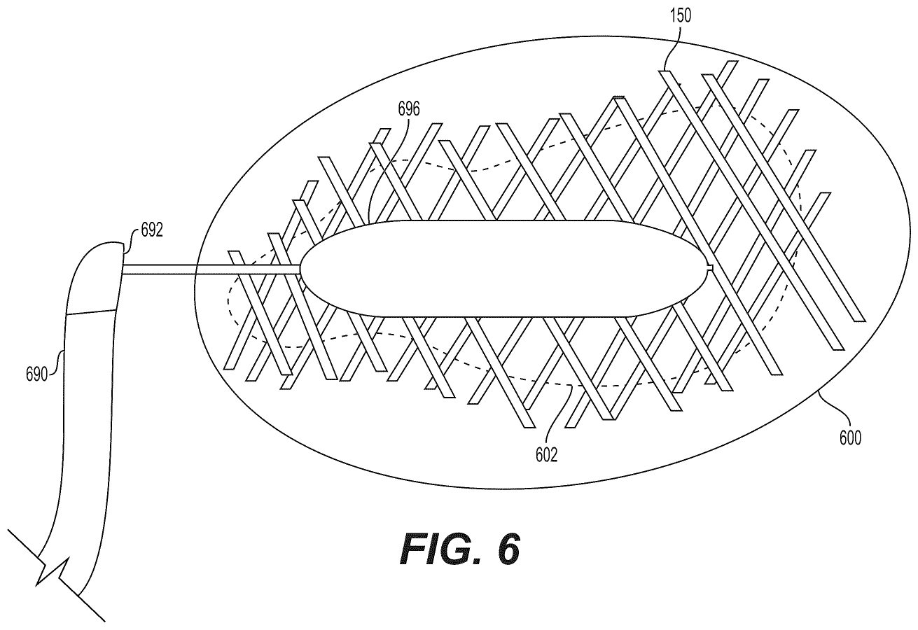

Medical devices for delivering a plurality of strips to a target site according to the present disclosure, e.g., delivery devices, may comprise a lumen and/or chamber for delivery of a plurality of strips made from a biocompatible material or combination of biocompatible materials as described herein. The delivery device may be inserted into a working channel of an endoscope. In some examples, the delivery device may include a shaft with a distal end in contact with the plurality of strips to push the plurality of strips out of a distal opening of the delivery device. For example, the plurality of strips may be housed within a chamber proximate the distal end of the delivery device and distal to a shaft capable of moving longitudinally along a lumen of the delivery device. As a user pushes the shaft distally, the distal end of the shaft may push the plurality of strips out of the chamber and through the distal opening. Kits and medical systems herein may comprise a device comprising a plurality of strips to be delivered to a subject (such devices also referred to herein as delivery devices), and optionally an endoscope or other medical device useful for advancing the delivery device to the target site. For example, an exemplary kit may comprise a first medical device housing a plurality of strips comprising a biocompatible material (e.g., pre-loaded with a plurality of strips) and a second medical device defining a working channel, the first device being insertable into the working channel. Exemplary first medical devices for such kits are illustrated in FIGS. 1A-1C, 3, and 4. Exemplary second medical devices for such kits are illustrated in FIGS. 5 and 6. The devices and kits comprising such devices herein are not limited to the devices and features thereof depicted in the figures; rather variations are contemplated and are described throughout the present disclosure.

FIG. 1A depicts an exemplary delivery device 100 according to some aspects of the present disclosure, wherein the device 100 is in an assembled state. Device 100 houses a plurality of strips 150 at distal portion 170 and includes a mechanism for delivering the strips 150 to a target site. The mechanism is depicted as including a shaft 120 with a distal end 130 proximate the strips 150. For example, the shaft 120 may be part of a plunger with a proximal handle 110 to allow a user to move the shaft 120 along a lumen of the device. The lumen may be defined by a catheter 140. The lumen may extend from a proximal portion of device 100 to a distal end of device 100, e.g., the lumen being in communication with a distal opening through which the strips 150 may exit the device 100. The distal portion of device 100 may be sized to fit within a working channel of an endoscope. For example, catheter 140 may have any suitable outer cross-sectional dimension that fits inside a working channel of an endoscope. Catheter 140 may be flexible so that the device 100 may be advanced through a working channel of an endoscope in a body lumen, which may have tortuous anatomy. The handle 110 may be operatively connected to a distal end 130 of the shaft 120. The shaft and distal end 130 thereof may be moveable along the lumen via manipulation of the handle 110.

The plurality of strips 150 comprise a biocompatible material, which may comprise chitosan in some examples. The plurality of strips 150 may be housed within the distal portion of the device 100, such as within a chamber proximate at the distal end of device 100. The plurality of strips 150 in device 100 may have a uniform length and/or width (e.g., the strips being rectangular or square in shape), or may vary in length and/or width. In some examples, the length of each strip 150 may range from about 5 mm to about 40 mm, and the width of each strip may range from about 1.5 mm to about 5 mm. For example, a plurality of strips having a length of about 20 mm, about 25 mm, and about 30 mm may be used to treat a target site having a cross-sectional dimension ranging from about 10 mm to about 20 mm (e.g., a perforation of about 15 mm in cross-sectional dimension). Each of the plurality of strips 150 may have a substantially flat or planar configuration when housed in device 100 prior to application to a target site. For example, each of the plurality of strips may be configured to avoid bending of each strip and/or bunching together of the plurality of strips when housed in device 100.

FIGS. 1B-1C depict components of delivery device 100. For example, FIG. 1B shows catheter 140 having a proximal end 160, a distal portion 170, and an outlet or distal opening 180. The distal portion 170 may contain the plurality of strips 150 as shown in FIG. 1A, such as within a chamber of device 100. The plurality of strips 150 may be preloaded in the distal portion 170 of device 100. A user may load a desired amount of the strips 150 into distal portion 170 prior to inserting the device 100 into an endoscope.

FIG. 1C shows the mechanism for delivering the strips 150, the mechanism including comprising the handle 110 and shaft 120 with distal end 130. For example, the mechanism may collectively form a plunger assembly capable of pushing or dispensing the plurality of strips 150 out of device 100. The distal end 130 of shaft 120 may be configured to be slidably housed within the lumen of the device 100. For example, the distal end 130 may be configured to move distally within the lumen of catheter 140. The handle 110 may be used to move the shaft 120 and distal end 130 thereof distally, towards the strips 150. The handle 110 and/or distal end 130 may be integral with the shaft 120 or may be operatively coupled to shaft 120 via, e.g., crimping, soldering, gluing, or other fixing mechanisms. Shaft 120 may be flexible while sufficiently rigid so as to transmit a force from movement of handle 110 to distal end 130 of shaft 120. However, shaft 120 may be sufficiently flexible so as to not inhibit or flexibility of catheter 140. For example, shaft 120 may comprise a material such as nitinol, stainless steel, plastic or other polymer, braided coil, Nylon-12 in a flexible configuration. In at least one example, shaft 120 comprises a wire, e.g., a Nitinol wire.

Device 100 may be inserted into a working channel of an endoscope or other delivery device so that the distal portion 170 of device 100 may be positioned proximate a target site of a tissue wall via the endoscope, e.g., tissue of the gastrointestinal tract. For example, a user may use the endoscope to position the working channel and device 100 therein at a target site. Once the distal portion 170 of device 100 is proximate the target site, the handle 110 may be used to advance the shaft 120 distally, which causes distal end 130 to advance within distal portion 170 towards the plurality of strips 150. The shaft 120 may thereof push the plurality of strips 150 through the distal opening 180. Pausing the advancement of shaft 120 distally or pulling shaft 120 proximally via the handle 110 may stop the dispensing of strips 150. In some examples, device 100 may be a device that is a part of a kit. For example, device 100 may be a first medical device in a kit that includes a second medical device. For example, the second medical device may be an endoscope. Device 100 may be inserted into a working channel of the endoscope from the kit for positioning at a target site.

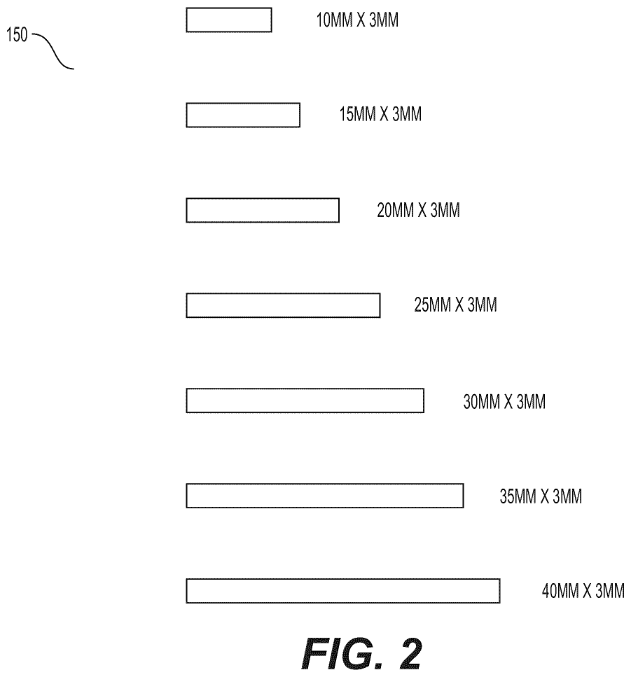

FIG. 2 shows exemplary dimensions (not shown to scale) for strips in accordance with some examples herein. For example, the plurality of strips 150 in FIG. 1A may include some or all of the exemplary strips with dimensions shown in FIG. 2, e.g., the plurality of strips 150 being substantially rectangular in shape. In some examples, the plurality of strips 150 may have a width ranging from about 1.5 mm to about 5 mm, such as a width of about 3 mm. The length of each strip 150 may range from about 10 mm to about, 40 mm, e.g., a length of about 10 mm, about 15 mm, about 20 mm, about 25 mm, about 30 mm, about 35 mm, or about 30 mm. In at least one example, each strip of the plurality of strips 150 may have the same length and width, e.g., a length of about 10 mm and a width of about 3 mm, or a length of about 40 mm and a width of about 3 mm. The dimensions of the plurality of strips 150 used in device 100 may be selected based on the size and shape of a tissue defect to be treated.

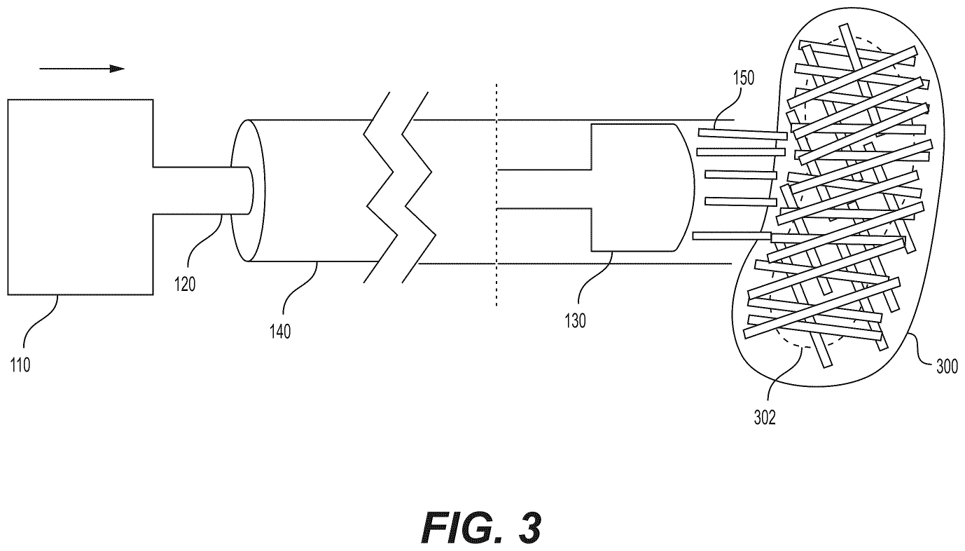

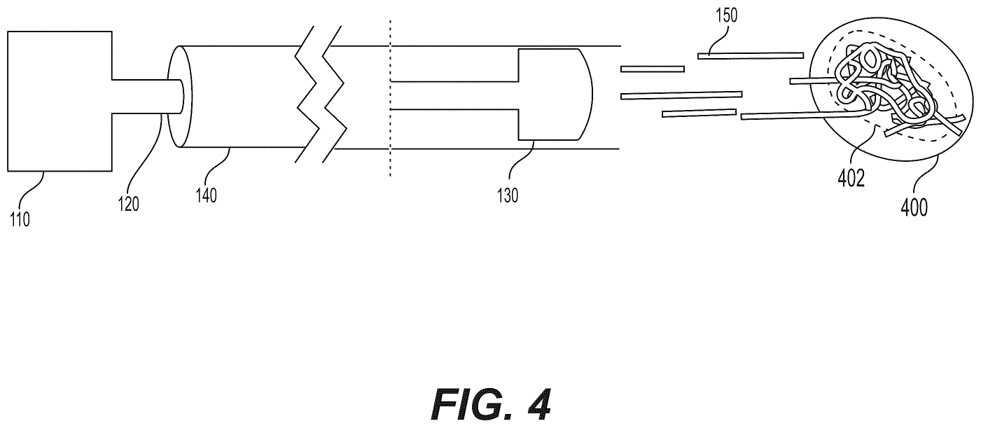

FIGS. 3 and 4 illustrate exemplary methods of delivering a plurality of strips 150 to a target site 300 and 400, respectively, according to some aspects of the present disclosure. Specifically, distal portion 170 of device 100 is positioned proximate to respective target sites 300, 400. For purposes of illustration herein, a partial thickness tissue defect may be distinguished from a full thickness tissue defect based on size. A partial thickness tissue defect may be displayed with a larger surface area and full thickness tissue defect may be displayed with a smaller surface area.

In FIG. 3, a plurality of strips 150 are shown housed within the distal portion 170 of device 100, proximate the distal opening of device 100 that is facing target site 300, containing tissue defect 302. FIG. 3 also depicts the distal end 130 of shaft 120 as being proximal to and proximate the plurality of strips 150. As handle 110 is actuated (e.g., advancing handle 110 in the distal direction toward the plurality of strips 150), the distal end 130 of shaft 120 is advanced distally to contact the plurality of strips 150. Distal end 130 is shown as pushing the plurality of strips out of catheter 140 and onto the target site 300, to contact a defect 302 of the target site 300. FIG. 3 shows the plurality of strips 150 being applied at the same time. The defect 302 of target site 300 may be a partial thickness tissue defect such that the defect or depth of the defect does not extend completely through the tissue. Partial thickness tissue defects, such as the defect depicted in FIG. 3, may be treated by covering or patching at the surface rather than a complete filling of the defect. As shown in FIG. 3, the plurality of strips 150 form a layer or barrier, having a two-dimensional surface over the defect 302 of target site 300. The plurality of strips 150 are depicted as forming an overlapping structure across the surface of target site 300 including the defect 302.

The plurality of strips 150 while housed within device 100 may maintain a flat, planar configuration. Upon application to target site 300, the plurality of strips 150 may be dispersed across the defect 302 at the surface of the target site 300. At least some of the plurality of strips 150 distributed at the defect 302 may overlap. The user may continue to apply the strips 150 by dispensing from the delivery device until the defect 302 is substantially or completely covered. The user may add additional strips 150 as desired or when necessary. In at least one example, when strips 150 of device 100 have been expended, a user may remove device 100 and replace it with another device that has been pre-loaded with a plurality of strips 150. In another example, when the strips 150 have been expended, the user may add additional strips 150. For example, the user may add more strips 150 at the proximal end of device 100 (optionally after removing the plunger assembly, e.g., plunger 110, shaft, 120, and distal end 130 of shaft 120), and pushing the strips 150 to the distal portion 170 of device 100 using the plunger assembly. In some examples, the additional strips 150 or a replacement device pre-loaded with a plurality of strips 150 may be included in a kit comprising device 100 and optionally another second medical device, such as an endoscope.

In FIG. 4, a plurality of strips 150 are applied to target site 400 having a defect 402, in a similar manner as shown in FIG. 3. Target site 400 may include a full thickness tissue defect, e.g., defect 402, that extends through the entire tissue wall of the target site 400. For example, the defect 402 may be a perforation. Such full thickness tissue defects may be treated with a plurality of strips 150 to extend into and at least partially fill the tissue defect. As shown in FIG. 4, the plurality of strips 150 are dispensed from the device 100 in a planar configuration. Each of the plurality of strips 150 adapt a curled or twisted configuration once applied to the tissue to fit into the defect 402 at target site 400. As depicted in FIG. 4, a change in configuration of the strips 150 may occur after the plurality of strips 150 exit the device 100 and contact the tissue. This change in configuration allows the strips 150 to fit inside certain tissue defects (e.g., full thickness tissue defects), rather than spread across the surface. The user may continue to dispense strips 150 from the device 100 until the defect 402 at target site 400 is substantially or completely filled by strips 150.

According to some aspects of the present disclosure, further steps may be performed after applying the plurality of strips 150 to the target site (e.g., onto a tissue defect) to promote adhesion and/or curling of the plurality of strips and to achieve the configuration for filling a tissue defect as shown in FIG. 4. Adhesion as well as a curling or twisting of a plurality of strips may be facilitated by spraying a liquid such as water or an aqueous solution (e.g., saline solution) onto the target site after the plurality of strips have been applied. The pH of the liquid may be varied in order to promote curling of the strips based on the biocompatible material of the strips. For example, the liquid may be water or an aqueous solution having an acidic or neutral pH. For example, chitosan materials generally include positively charged groups (e.g., protonated amine groups). When the plurality of strips comprise chitosan or derivative thereof, water or an aqueous solution with an acidic pH of less than 7 may be applied onto the target site after the strips have been applied to promote curling of the strips. In some examples, pure water having a pH of about 7, may be applied to strips that are made from biocompatible materials, such as poly(HEMA), which may exhibit curling in a neutral pH system.

In some examples, at least one or all of the strips applied to the target site with the device may have variable thickness along the length and/or width of the strip(s). Such dimensions may cause the strip(s) to curl upon contact with moisture at the target site and/or upon the application of a liquid, such as water or aqueous solution, to the target site to which the strips have been applied. For example, when water or aqueous solution is applied to a plurality of strips comprising an adsorbent and/or hydrophilic material, the strips may swell and the variable thickness may cause each strip to curl at different points upon the absorption of water. The water or aqueous solution may additionally or alternatively have a pH to promote curling. For instance, a strip comprising chitosan with positively-charged groups and having a uniform thickness may curl upon the application of acidic water or aqueous solution but not by application of water or aqueous solution having a neutral or basic pH, while a strip comprising chitosan with positively charged groups and having a variable thickness may curl upon the application of water or aqueous solution at neutral pH.

In examples herein, the liquid (e.g., water or aqueous solution) may be applied to the plurality of strips at the target site as a spray (e.g., mist). For example, after the plurality of strips 150 are applied to the target site (e.g., onto a tissue defect), a source of liquid (e.g., water or aqueous solution) may be applied directly to the strips to facilitate adhesion of the strips and interaction between the plurality of strips at the target site. The amount of liquid applied may be selected based at least partially on the thickness of the plurality of strips.

FIGS. 5 and 6 show exemplary methods of promoting adhesion of a plurality of strips to target tissue sites 500 and 600, respectively, after the strips have been applied (e.g., using device 100 of FIG. 1A, such as depicted in FIG. 3). For example, FIGS. 5 and 6 each depict a plurality of strips 150 that have been applied across a tissue defect. FIG. 5 shows a plurality of strips 150 forming a layer or barrier comprising overlapping strips 150 over a defect 502 of a target site 500. The defect 502 at target site 500 may be a partial thickness tissue defect such that the depth of the defect does not extend completely through the tissue wall. Similarly, FIG. 6 shows a plurality of strips 150 that have been applied onto a defect 602 of a target site 600 in an overlapping configuration to provide a layer or barrier. The defect 602 of target site 600 may also be a partial thickness tissue defect.

In FIG. 5, after the plurality of strips 150 are applied onto the defect 502 of target site 500, a liquid 594 may be applied to the plurality of strips 150 to promote adhesion to the tissue of target site 500. The liquid 594 may be water or an aqueous solution. The liquid 594 may be sprayed or otherwise applied to target site 500 using a suitable device such as an endoscope 590 to contact the plurality of strips 150 at target site 500. The liquid 594 may be dispensed through a working channel 592 of endoscope 590. The endoscope 590 also may be used to apply the strips 150 in prior steps, via a device 100 through the same working channel 592 or another channel, e.g., fluid channel. In some examples, endoscope 590 may be part of a kit that also includes another medical device for applying the plurality of strips to the target site, such as device 100.

In FIG. 6, after the plurality of strips 150 have been applied onto a defect 602 of target site 600, an inflatable device, e.g., balloon of balloon device 694 may be deployed and applied on top of the plurality of strips to contact the strips 150 and promote adhesion to the tissue. In some examples, the balloon may be coupled to endoscope 690 rather than part of a balloon device 694 delivered to the target site 602 via a working channel 692 as shown. The balloon may be in communication with a source of fluid such as air or water for inflation and deflation. Upon inflation at the target site, the balloon may contact, e.g., lightly press against, the strips 150 at the target site 600 to promote adhesion of the plurality of strips 150 to the tissue. In at least one example, endoscope 690 may be part of a kit that also includes another medical device for applying the plurality of strips to the target site, such as device 100. Optionally, the kit may also include a balloon device such as balloon device 694.

In some examples, both liquid and a balloon may be applied onto a plurality of strips applied to a tissue defect. Applying both water or other suitable liquid and a balloon may aid in increasing adhesion of the strips to the tissue. The application of liquid and/or a balloon for the purpose of promoting adhesion may be performed at sites where the plurality of strips have been applied to partial thickness tissue defects. In some examples, water or other suitable liquid may be applied to the strips at the target site prior to the application of a balloon.

The present disclosure also includes methods of filling, patch, and/or covering a tissue defect, e.g., at a target tissue site, with a plurality of strips comprising a biocompatible material followed by the application of one or more therapeutic agents to the target site. Exemplary therapeutic agents useful for the present disclosure include, but are not limited to, hemostatic agents, antibacterial agents, coagulation cascade activators, growth factors, anti-inflammatory agents, and cancer therapies. The hemostatic agent may be natural or derived from natural materials, or may be at least partially synthetic in origin. For example, the hemostatic agent may comprise one or more polysaccharides such as, e.g., chitosan, natural gums, alginate, cellulose, starch (e.g., potato or other plant starch), and glycogen. In at least one example, the hemostatic agent may be charged, e.g., cationic. In some examples, the hemostatic agent may be in particle form, e.g., formulated as a powder, or may be liquid form, e.g., formulated as a spray such as with a biocompatible liquid.

In some embodiments, the hemostatic agent or other therapeutic agent may be applied to the tissue defect via spraying. For example, the therapeutic agent may be sprayed onto the target site that includes the plurality of strips previously applied to the tissue defect. In examples where a balloon is used to promote adhesion of the plurality of strips to the tissue, the balloon may be deflated and removed from the site before applying the therapeutic agent.

The therapeutic agent may be applied to the target site any time after the plurality of strips have been applied to the target site. In some examples, the therapeutic agent may be applied within seconds to minutes of applying the plurality of strips, e.g., within about 10 minutes, about 5 minutes, about 1 minute, about 30 seconds, or about 5 seconds, e.g., from 1 second to 10 minutes, from 5 seconds to 5 minutes, or from 10 seconds to 30 seconds.

The application of a plurality of strips to a tissue defect in accordance with aspects of the present disclosure may aid in preventing undesired substances from passing through the tissue defect. The devices and methods of the preset disclosure may allow for application of a therapeutic agent such as a hemostatic agent at a target site without the therapeutic agent passing through a tissue defect site to unwanted parts of the body.

Other embodiments of the present disclosure will be apparent to those skilled in the art from consideration of the specification and practice of the embodiments disclosed herein. It is intended that the specification and examples be considered as exemplary only, with a true scope and spirit of the present disclosure being indicated by the following claims.

We claim:

1. A method of treating a subject, the method comprising:
introducing a distal portion of a medical device into a gastrointestinal tract of the subject, wherein the distal portion houses a plurality of strips comprising a biocompatible material;
positioning the distal portion of the device proximate a target site of a tissue wall of the gastrointestinal tract that includes a defect; and
applying the plurality of strips to the target site by pushing the plurality of strips out of a distal opening of the device, wherein the plurality of strips at least partially fill the defect or form a layer over the defect.

2. The method of claim 1, wherein the biocompatible material comprises chitosan, cellulose, poly(2-hydroxyethyl methacrylate), polystyrene, collagen, gelatin, fibrin, polyethylene glycol (PEG), hyaluronic acid, a block copolymer, or a combination thereof.

3. The method of claim 2, wherein the biocompatible material comprises, thiolated chitosan, chitosan crosslinked with tripolyphosphate, or carboxymethylcellulose chitosan crosslinked with PEGamine.

4. The method of claim 1, wherein the biocompatible material is bioresorbable.

5. The method of claim 1, further comprising applying a therapeutic agent to the target site after applying the plurality of strips to the target site.

6. The method of claim 5, wherein the therapeutic agent is a hemostatic agent.

7. The method of claim 5, wherein applying the therapeutic agent comprises spraying the therapeutic agent onto the target site.

8. The method of claim 1, wherein pushing the plurality of strips out of the distal opening of the device includes advancing a shaft of the device distally to push the plurality strips with a distal end of the shaft.

9. The method of claim 8, wherein the distal end of the shaft has a cross-sectional dimension larger than a cross-sectional dimension of the shaft, such that the distal end of the shaft contacts and slides along walls of a lumen as the shaft moves along the lumen.

10. The method of claim 1, wherein each strip of the plurality of strips has a length ranging from about 5 mm to about 40 mm, a width ranging from about 1.5 mm to about 5 mm, or a thickness ranging from about 1 μm to about 100 μm.

11. The method of claim 1, further comprising applying a biocompatible liquid or a balloon to the plurality of strips after applying the plurality of strips to the target site.

12. The method of claim 1, wherein the plurality of strips change from a planar configuration when housed within the device to a curled configuration when applied to the target site.

13. The method of claim 1, wherein the plurality of strips includes at least one strip having a variable thickness along a length of the at least one strip.

14. A method of treating a subject, the method comprising:
introducing a distal portion of a medical device into a gastrointestinal tract of the subject, wherein the distal portion of the device defines a lumen that houses a shaft and a chamber proximate a distal end of the device, wherein the chamber houses a plurality of strips comprising a biocompatible material;
positioning the distal portion of the device proximate a target site of a tissue wall that includes a defect;
advancing the shaft distally to push the plurality of strips out of a distal opening of the device and onto the target site; and
applying a biocompatible liquid, a therapeutic agent, or a balloon to the target site after applying the plurality of strips.

15. The method of claim 14, wherein the biocompatible material comprises chitosan, cellulose, poly(2-hydroxyethyl methacrylate), polystyrene, collagen, gelatin, fibrin, polyethylene glycol (PEG), hyaluronic acid, a block copolymer, or a combination thereof.

16. The method of claim 14, wherein the biocompatible material is bioresorbable, a hemostatic material, or both.

17. The method of claim 16, wherein the method includes applying a therapeutic agent to the target site after applying the plurality of strips, the therapeutic agent being a hemostatic agent.

18. A method of treating a subject, the method comprising:
introducing a distal portion of a medical device into a gastrointestinal tract of the subject, wherein the distal portion of the device includes a chamber that houses a plurality of strips comprising chitosan or derivative thereof;
positioning the distal portion of the device proximate a target site of a tissue wall of the gastrointestinal tract that includes defect; and
applying the plurality of strips to the target site by pushing the plurality of strips out of a distal opening of the device;
wherein each strip of the plurality of strips has a variable thickness along a length of the strip, and the plurality of strips change from a planar configuration when housed within the device to a curled configuration when applied to the target site.

19. The method of claim 18, further comprising applying a biocompatible liquid, a therapeutic agent, or a balloon to the target site after applying the plurality of strips to the target site.

20. The method of claim 19, wherein the method includes spraying water or an aqueous solution having an acidic pH onto the target site after applying the plurality of strips to the target site.
